(19) United States
(12) Patent Application Publication
(10) Pub. No.: US 2009/0306458 A1
(43) Pub. Date: Dec. 10, 2009

(54) Direct acoustic cochlear stimulator for round window access

(75) Inventors: John L. Parker, Roseville (AU); Thomas Lenarz, (US); Markus Halier, (US)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/349,502

(22) Filed: Jan. 6, 2009

Related U.S. Application Data
(60) Provisional application No. 61/041,185, filed on Mar. 31, 2008.

(51) Int. Cl. A61F 11/04; H04R 25/00

(52) U.S. Cl. 600/25

(57) ABSTRACT

A mechanical stimulator for evoking a hearing percept by directly generating waves of fluid motion of fluid in a recipient's scala tympani. The stimulator comprises a sound processing unit configured to process a received sound signal; and an implantable stimulation arrangement, comprising: an actuator configured to receive electrical signals representing the processed sound signal and configured to vibrate in response to the electrical signals, a stapes prosthesis having first and second ends, the first end having a surface configured to be positioned abutting the round window in the recipient's cochlea, and wherein the first end surface is substantially orthogonal to a longitudinal axis extending through the actuator, an elongate rod extending longitudinally from the actuator connecting the actuator to the stapes prosthesis such that vibration of the actuator results in waves of fluid motion in a recipient's scala tympani that evoke a hearing percept of the received sound signal.

### CROSS-REFERENCE TO RELATED APPLICATIONS

The present application claims the benefit of U.S. Provisional Patent Application 61/041,185; filed Mar. 31, 2008, which is hereby incorporated by reference herein. Furthermore, this application is a related to commonly owned and co-pending U.S. patent application entitled “MECHANICAL SEMICIRCULAR CANAL STIMULATOR,” filed concurrently herewith under Attorney Docket No. 22409-00498-US. This application is hereby incorporated by reference herein in its entirety.

### BACKROUND

1. Field of the Invention

The present invention is related to a hearing prosthesis, and particularly to, a mechanical scala tympani stimulator.

2. Related Art

Hearing loss, which may be due to many different causes, is generally of two types, conductive and sensorineural. In some cases, an individual may have hearing loss of both types. In many people who are profoundly deaf, however, the reason for their deafness is sensorineural hearing loss. Sensorineural hearing loss occurs when there is damage to the inner ear, or to the nerve pathways from the inner ear to the brain. As such, those suffering from sensorineural hearing loss are thus unable to derive suitable benefit from conventional acoustic hearing aid. As a result, hearing prostheses that deliver electrical stimulation to nerve cells of the recipient's auditory system have been developed to provide persons having sensorineural hearing loss with the ability to perceive sound. Such electrically-stimulating hearing prostheses deliver electrical stimulation to nerve cells of the recipient's auditory system.

As used herein, the recipient's auditory system includes all sensory system components used to perceive a sound signal, such as hearing sensation receptors, neural pathways, including the auditory nerve and spiral ganglion, and parts of the brain used to sense sounds. Electrically-stimulating hearing prostheses include, for example, auditory brain stimulators and Cochlear™ prostheses (commonly referred to as Cochlear™ prosthetic devices, Cochlear™ implants, Cochlear™ devices, and the like; simply “cochlear implants” herein.)

Most sensorineural hearing loss is due to the absence or destruction of the cochlea hair cells which transduce acoustic signals into nerve impulses. It is for this purpose that cochlear implants have been developed. Cochlear implants use direct electrical stimulation of auditory nerve cells to bypass absent or defective hair cells that normally transduce acoustic vibrations into neural activity. Such devices generally use an electrode array implanted in the cochlea so that the electrodes may differentially activate auditory neurons that normally encode differential pitches of sound.

In contrast to sensorineural hearing loss which results from damage to the inner ear, conductive hearing loss occurs when the normal mechanical pathways used to provide sound to hair cells in the cochlea are impeded, for example, by damage to the ossicular chain or to the ear canal. Individuals who suffer from conductive hearing loss typically have some form of residual hearing because the hair cells in the cochlea are undamaged. Such individuals are typically not candidates for a cochlear implant due to the irreversible nature of the cochlear implant. Specifically, insertion of the electrode array into a recipient's cochlea exposes the recipient to the risk of destruction of the majority of the hair cells within the cochlea, resulting in the loss of all residual hearing by the recipient.

As a result, individuals suffering from conductive hearing loss typically receive an acoustic hearing aid. Unfortunately, not all individuals who suffer from conductive hearing loss are able to derive suitable benefit from hearing aids. For example, some individuals are prone to chronic inflammation or infection of the ear canal and cannot wear hearing aids. Similarly, hearing aids are typically unsuitable for individuals who have malformed, damaged or absent outer ears, ear canals and/or ossicular chains.

### SUMMARY

In one aspect of the invention, a mechanical stimulator for evoking a hearing percept by directly generating waves of fluid motion of fluid in a recipient's scala tympani is provided. The stimulator comprises a sound processing unit configured to process a received sound signal; and an implantable stimulation arrangement, comprising: an actuator configured to receive electrical signals representing the processed sound signal and configured to vibrate in response to the electrical signals, a stapes prosthesis having first and second ends, the first end having a surface configured to be positioned abutting the round window in the recipient's cochlea, and wherein the first end surface is substantially orthogonal to a longitudinal axis extending through the actuator, an elongate rod extending longitudinally from the actuator connecting the actuator to the stapes prosthesis such that vibration of the actuator results in waves of fluid motion in a recipient's scala tympani that evoke a hearing percept of the received sound signal.

In another aspect of the present invention, a system for rehabilitating the hearing of a recipient is provided. The system comprises: a sound processing unit configured to process a received sound signal; an actuator configured to receive electrical signals representing the processed sound signal and configured to vibrate in response to the electrical signals; a stapes prosthesis having a first end configured to be positioned abutting the round window in a recipient's cochlea; an elongate rod extending from the actuator; and a fixation system configured to be attached to the actuator and configured to position the actuator such that the coupler connects the actuator to the stapes prosthesis so that vibration of the actuator results in waves of fluid motion in the recipient's semicircular canal that evoke a hearing percept of the received sound signal.

In a still other aspect of the present invention, a method for rehabilitating the hearing of a recipient using a mechanical stimulator comprising a sound input element, a sound processing unit and an implantable stimulation arrangement is provided. The method comprises receiving at the sound input element an acoustic sound signal; converting with the sound processing unit the received sound signal into encoded data signals representing the received sound signal; providing the encoded data signals to the implantable stimulation arrangement; and generating with the implantable stimulation arrangement waves of fluid motion in a recipient's scala tympani that evoke a hearing percept of the received sound signal.

### BRIEF DESCRIPTION OF THE FIGURES

Illustrative embodiments of the present invention are described herein with reference to the accompanying drawings, in which:

FIG. 1A is a partial cross-sectional view of an individual's head;

FIG. 1B is a perspective, partially cut-away view of a cochlea exposing the canals and nerve fibers of the cochlea;

FIG. 1C is a cross-sectional view of one turn of the canals of a human cochlea;

FIG. 2A is a perspective view of a direct mechanical stimulator in accordance with embodiments of the present invention shown implanted in a recipient;

FIG. 2B is a perspective view of a direct mechanical stimulator in accordance with embodiments of the present invention shown implanted in a recipient;

FIG. 3 is a partially exploded top view of a direct mechanical stimulator, in accordance with embodiments of the present invention;

FIG. 4A is a perspective view of a stimulation arrangement, in accordance with embodiments of the present invention;

FIG. 4B is a perspective view of a first component of a coupler, in accordance with embodiments of the present invention;

FIG. 4C is a cross-sectional view of a second component of a coupler, in accordance with embodiments of the present invention;

FIG. 5A is a perspective view of a portion of an implanted component of a direct mechanical stimulator, in accordance with embodiments of the present invention;

FIG. 5B a perspective view of a portion of an implanted component of a direct mechanical stimulator, in accordance with alternative embodiments of the present invention;

FIG. 5C is a perspective view of a stapes prosthesis, in accordance with embodiments of the present invention;

FIG. 5D is a cross-sectional side view of a stapes prosthesis, in accordance with embodiments of the present invention;

FIG. 6 is a functional block diagram of a direct mechanical stimulator, in accordance with embodiments of the present invention; and

FIG. 7 is a perspective view of a fixation system implemented in conjunction with a direct mechanical stimulator, in accordance with embodiments of the present invention

### DETAILED DESCRIPTION

Aspects of the present invention are generally directed to a hearing prosthesis which simulates natural hearing by generating mechanical motion of the fluid within a recipient's cochlea. Such a hearing prosthesis, referred to herein as direct mechanical stimulator, bypasses the recipient's outer and middle ears to directly generate waves of fluid motion of the cochlear fluid, thereby activating cochlear hair cells and evoking a hearing percept

Specifically, a direct mechanical stimulator in accordance with embodiments of the present invention comprises a stapes prosthesis abutting an opening in the recipient's inner ear. Coupled to the stapes prosthesis is an implanted actuator which is configured to vibrate the stapes prosthesis. The vibration of the stapes prosthesis generates the waves of fluid motion of the cochlear fluid.

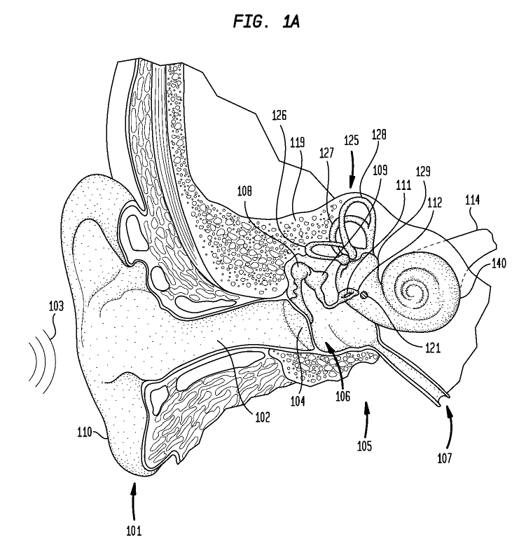

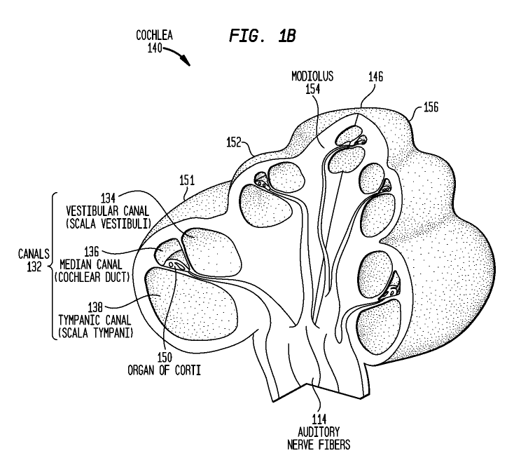

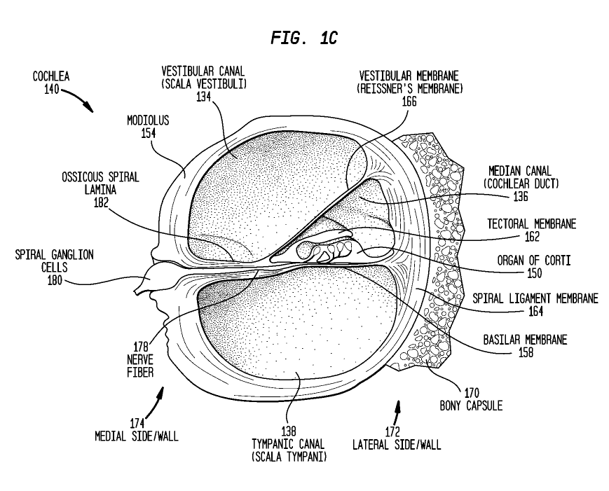

FIG. 1A is perspective view of an individual's head in which a direct mechanical stimulator in accordance with embodiments of the present invention may be implemented. As shown in FIG. 1A, the individual's hearing system comprises an outer ear 101, a middle ear 105 and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A are semicircular canals 125. Semicircular canals 125 are three half-circular, interconnected tubes located adjacent cochlea 140. The three canals are the horizontal semicircular canal 126, the posterior semicircular canal 127, and the superior semicircular canal 128. The canals 126, 127 and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the skull twists in any direction, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

The details of cochlea 140 are described next below with reference to FIGS. 1B and 1C. FIG. 1B is a perspective view of cochlea 140 partially cut-away to display the canals and nerve fibers of the cochlea. FIG. 1C is a cross-sectional view of one turn of the canals of cochlea 140.

Referring to FIG. 1B, cochlea 140 is a conical spiral structure comprising three parallel fluid-filled canals or ducts, collectively and generally referred to herein as canals 132. Canals 132 comprise the tympanic canal 138, also referred to as the scala tympani 138, the vestibular canal 134, also referred to as the scala vestibuli 134, and the median canal 136, also referred to as the cochlear duct 136. Cochlea 140 has a conical shaped central axis, the modiolus 154, that forms the inner wall of scala vestibuli 134 and scala tympani 138. The base of scala vestibuli 134 comprises oval window 112 (FIG. 1A), while the base of scala tympani 138 terminates in round window 121 (FIG. 1A). Tympanic and vestibular canals 138, 134 transmit pressure waves received at oval window 112, while medial canal 136 contains the organ of Corti 150 which detects pressure impulses and responds with electrical impulses which travel along auditory nerve 114 to the brain (not shown).

Cochlea 140 spirals about modiolus 154 several times and terminates at cochlea apex 146. Modiolus 154 is largest near its base where it corresponds to first turn 151 of cochlea 140. The size of modiolus 154 decreases in the regions corresponding to medial 152 and apical turns 156 of cochlea 140.

Referring now to FIG. 1C, separating canals 132 of cochlear 140 are various membranes and other tissue. The Ossicous spiral lamina 182 projects from modiolus 154 to separate scala vestibuli 134 from scala tympani 138. Toward lateral side 172 of scala tympani 138, a basilar membrane 158 separates scala tympani 138 from median canal 136. Similarly, toward lateral side 172 of scala vestibuli 134, a vestibular membrane 166, also referred to as the Reissner's membrane 166, separates scala vestibuli 134 from median canal 136.

Portions of cochlea 140 are encased in a bony capsule 170. Bony capsule 170 resides on lateral side 172 (the right side as drawn in FIG. 1C), of cochlea 140. Spiral ganglion cells 180 reside on the opposing medial side 174 (the left side as drawn in FIG. 1C) of cochlea 140. A spiral ligament membrane 164 is located between lateral side 172 of spiral tympani 138 and bony capsule 170, and between lateral side 172 of median canal 136 and bony capsule 170. Spiral ligament 164 also typically extends around at least a portion of lateral side 172 of scala vestibuli 134.

The fluid in tympanic and vestibular canals 138, 134, referred to as perilymph, has different properties than that of the fluid which fills median canal 136 and which surrounds organ of Corti 150, referred to as endolymph. Sound entering auricle 110 causes pressure changes in cochlea 140 to travel through the fluid-filled tympanic and vestibular canals 138, 134. As noted, organ of Corti 150 is situated on basilar membrane 158 in median canal 136. It contains rows of 16,000-20,000 hair cells (not shown) which protrude from its surface. Above them is the tectoral membrane 162 which moves in response to pressure variations in the fluid-filled tympanic and vestibular canals 138, 134. Small relative movements of the layers of membrane 162 are sufficient to cause the hair cells to send a voltage pulse or action potential down the associated nerve fiber 178. Nerve fibers 178, embedded within spiral lamina 182, connect the hair cells with the spiral ganglion cells 180 which form auditory nerve 114. Auditory nerve 114 relays the impulses to the auditory areas of the brain (not shown) for processing.

As described above with reference to FIG. 1A, semicircular canals 125 are also filled with endolymph. The vestibule 129 (FIG. 1A) provides fluid communication between the endolymph in semicircular canals 125 and the endolymph in median canal 136.

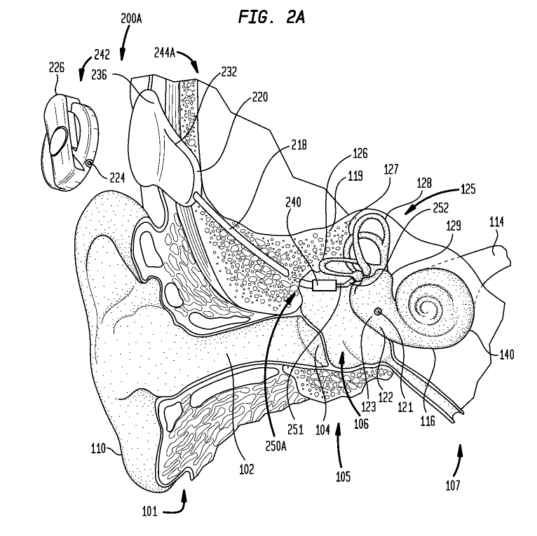

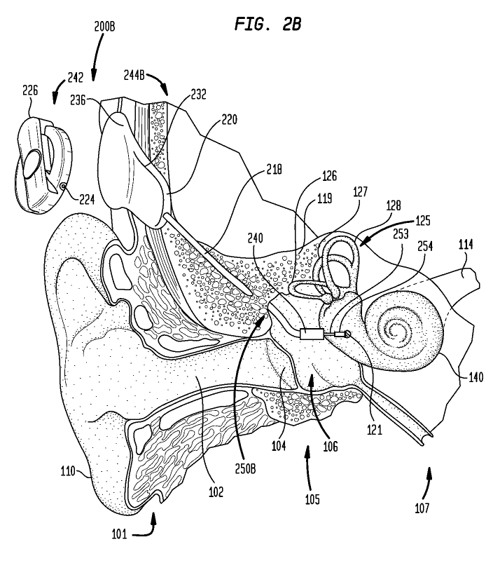

FIG. 2A is a perspective view of a direct mechanical stimulator 200A in accordance with embodiments of the present invention having Direct mechanical stimulator 200A is shown have components implanted in a recipient.

Direct mechanical stimulator 200A comprises an external component 242 which is directly or indirectly attached to the body of the recipient, and an internal component 244A which is temporarily or permanently implanted in the recipient. External component 242 typically comprises one or more sound input elements, such as microphones 224 for detecting sound, a sound processing unit 226, a power source (not shown), and an external transmitter unit (also not shown). The external transmitter unit is disposed on the exterior surface of sound processing unit 226 and comprises an external coil (not shown). Sound processing unit 226 processes the output of microphones 224 and generates encoded signals, sometimes referred to herein as encoded data signals, which are provided to the external transmitter unit. For ease of illustration, sound processing unit 226 is shown detached from the recipient.

Internal component 244A comprises an internal receiver unit 232, a stimulator unit 220, and a stimulation arrangement 250A. Internal receiver unit 232 and stimulator unit 220 are hermetically sealed within a biocompatible housing, sometimes collectively referred to herein as a stimulator/receiver unit.

Internal receiver unit 232 comprises an internal coil (not shown), and preferably, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated single-strand or multi-strand platinum or gold wire. The electrical insulation of the internal coil is provided by a flexible silicone molding (not shown). In use, implantable receiver unit 132 may be positioned in a recess of the temporal bone adjacent auricle 110 of the recipient.

In the illustrative embodiment, stimulation arrangement 250A is implanted in middle ear 105. For ease of illustration, ossicles 106 have been omitted from FIG. 2A. However, it should be appreciated that stimulation arrangement 250A may be implanted without disturbing ossicles 106.

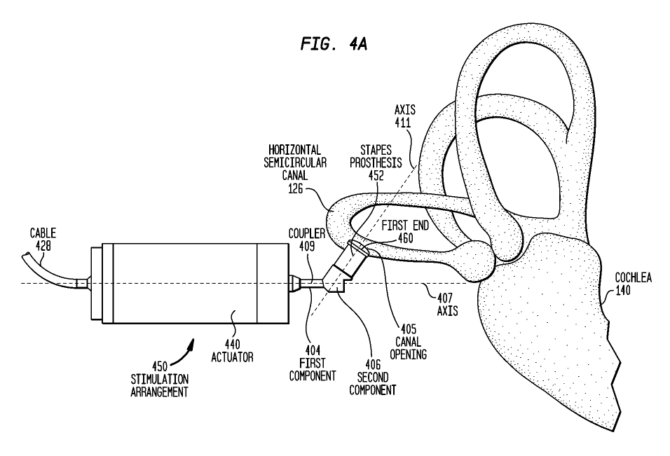

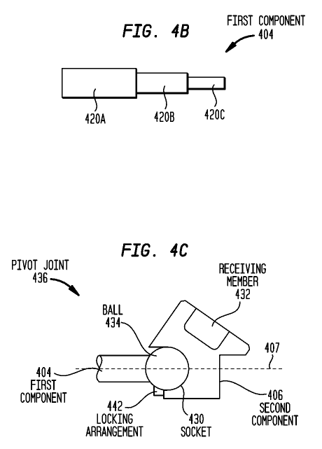

Stimulation arrangement 250A comprises an actuator 240, a stapes prosthesis 252 and a coupling element 251. As described in greater detail below with reference to FIGS. 4A and 4B, in this embodiment stimulation arrangement 250A is implanted and/or configured such that a portion of stapes prosthesis 252 abuts an opening in one of the semicircular canals 125. In the illustrative embodiment, stapes prosthesis 252 abuts an opening in horizontal semicircular canal 126. It would be appreciated that in alternative embodiments, stimulation arrangement 250A may be implanted such that stapes prosthesis 252 abuts an opening in posterior semicircular canal 127 or superior semicircular canal 128.

As noted above, a sound signal is received by one or more microphones 224, processed by sound processing unit 226, and transmitted as encoded data signals to internal receiver 232. Based on these received signals, stimulator 220 generates drive signals which cause actuation of actuator 240. This actuation is transferred to stapes prosthesis 252 such that a wave of fluid motion is generated in horizontal semicircular canal 126. Because, as noted above, vestibule 129 provides fluid communication between the semicircular canals 125 and the median canal 136 (FIG. 1B), the wave of fluid motion continues into median canal 136, thereby activating the hair cells of the organ of Corti 150 (FIG. 1C). Activation of the hair cells causes appropriate nerve impulses to be generated and transferred through the spiral ganglion cells (not shown) and auditory nerve 114 to the brain (also not shown) where they are perceived as sound.

FIG. 2B is a perspective view of a direct mechanical stimulator 200B in accordance with further embodiments of the present invention having Similar to the embodiments described above, direct mechanical stimulator 200B is shown have components implanted in a recipient.

Direct mechanical stimulator 200B comprises an external component 242 which is directly or indirectly attached to the body of the recipient, and an internal component 244B which is temporarily or permanently implanted in the recipient. As described above with reference to FIG. 2A, external component 242 typically comprises one or more sound input elements, such as microphones 224, a sound processing unit 226, a power source (not shown), and an external transmitter unit (also not shown). Also as described above, internal component 244B comprises an internal receiver unit 232, a stimulator unit 220, and a stimulation arrangement 250B.

In the illustrative embodiment, stimulation arrangement 250B is implanted in middle ear 105. For ease of illustration, ossicles 106 have been omitted from FIG. 2B. However, it should be appreciated that stimulation arrangement 250B may be implanted without disturbing ossicles 106.

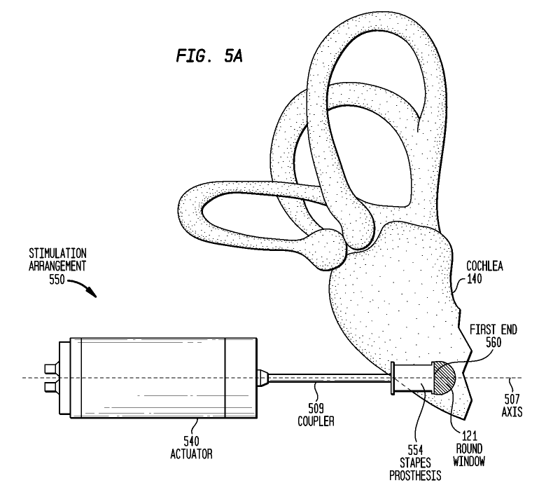

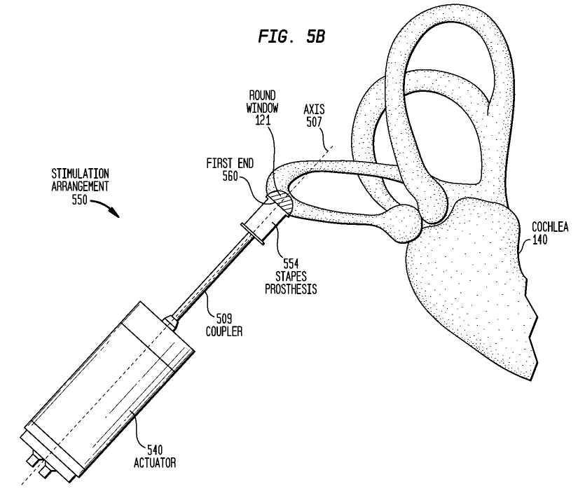

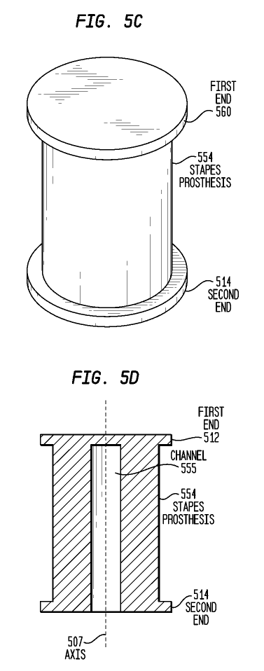

Stimulation arrangement 250B comprises an actuator 240, a stapes prosthesis 254 and a coupling element 253 connecting the actuator to the stapes prosthesis. As described in greater detail below with reference to FIGS. 5A-5C, in this embodiment stimulation arrangement 250B is implanted and/or configured such that a portion of stapes prosthesis 254 abuts round window 121 (FIG. 1A).

As noted above, a sound signal is received by one or more microphones 224, processed by sound processing unit 226, and transmitted as encoded data signals to internal receiver 232. Based on these received signals, stimulator 220 generates drive signals which cause actuation of actuator 240. This actuation is transferred to stapes prosthesis 254 such that a wave of fluid motion is generated in the perilymph in scala tympani 138 (FIG. 1B). Such fluid motion, in turn, activates the hair cells of the organ of Corti 150 (FIG. 1C). Activation of the hair cells causes appropriate nerve impulses to be generated and transferred through the spiral ganglion cells (not shown) and auditory nerve 114 to the brain (also not shown) where they are perceived as sound.

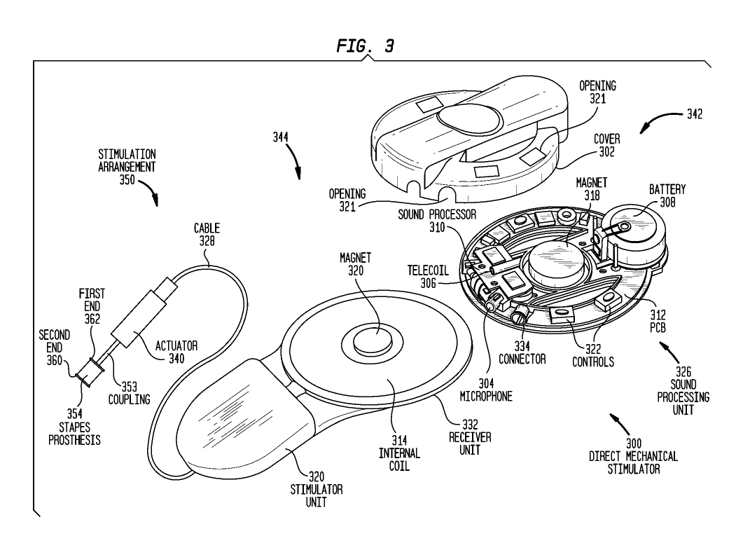

FIG. 3 is a partially exploded top view of a direct mechanical stimulator 300, in accordance with embodiments of the present invention. As discussed above, direct mechanical stimulator 300 comprises an external component 342 and an internal component 344. External component 342 comprises a sound processing unit 326. Disposed in or on sound processing unit 326 are one or more sound input elements configured to receive an input sound signal. In the illustrative embodiment of FIG. 3, sound processing unit 326 has microphones 324 disposed therein to receive an acoustic sound signal. Sound processing unit 326 further comprises an electrical connector 334. Electrical connector 334 is configured to connect mechanical stimulator 300 to external equipment, and to receive an electrical signal, such as an electrical sound signal, directly there from. Electrical connector 334 provides the ability to connect direct mechanical stimulator 300 to, for example, FM hearing systems, MP3 players, televisions, mobile phones, etc. Direct mechanical stimulator 300 further includes a sound input element in the form of a telecoil 306. Telecoil 306 provides the ability to receive input sound signals from, for example, a telephone or other similar device.

Sound processing unit 326 includes a sound processor 310 which processes sound signals received by the sound input elements. Sound processor 310 generates encoded data signals based on these received sound signals. To provide control over the sound processing and other functionality of direct mechanical stimulator 300, sound processing unit 326 includes one or more user controls 322. Integrated in sound processing unit 326 is a battery 308 which provides power to the other components of direct mechanical stimulator 300. Sound processing unit 326 further includes a printed circuit board (PCB) 312 to mechanically support and electrically connect the above and other functional components. Disposed on the exterior surface of sound processing unit 326 is an external transmitter unit (not shown).

For ease of illustration, sound processing unit 326 has been shown with cover 302 removed. Cover 302 further has one or more openings 321 therein which receive user controls 322, microphones 304 and connector 334. Cover 302 is configured to seal sound processing unit 326 so as to prevent the ingress of water, dust and other debris, particularly through openings 321.

Internal component 344 comprises an internal receiver unit 332, a stimulator unit 320, and a stimulation arrangement 350. As shown, receiver unit 232 comprises an internal coil 314, and preferably, a magnet 320 fixed relative to the internal coil. The external transmitter unit in external component 344 transmits electrical signals (i.e., power and stimulation data) to internal coil 314 via a radio frequency (RF) link. Signals received at internal coil 314 may be provided to stimulator unit 320. As would be appreciated, internal receiver unit 332 and stimulator unit 320 would be hermetically sealed within a biocompatible housing. This housing has been omitted from FIG. 3 for ease of illustration.

Connected to stimulator unit 320 via a cable 328 is a stimulation arrangement 350. Stimulation arrangement 350 comprises an actuator 340, a stapes prosthesis 354 and a coupling element 353. A second end of stapes prosthesis 354 is configured to be positioned abutting an opening in a recipient's inner ear. A second end of stapes prosthesis 354 is connected to an actuator 340 via a coupling 353. As described above, actuation of actuator vibrates stapes prosthesis 354. The vibration of stapes prosthesis 354 generates waves of fluid motion of the cochlear fluid, thereby activating the hair cells of the organ of Corti 150 (FIG. 1C). Activation of the hair cells causes appropriate nerve impulses to be generated and transferred through the spiral ganglion cells (not shown) and auditory nerve 114 to the brain (also not shown) where they are perceived as sound.

FIG. 4A illustrates a stimulation arrangement 450 in accordance with embodiments of the present invention. In the illustrative embodiment of FIG. 4A, stimulation arrangement 450 is configured to generate fluid motion of the endolymph contained in a recipient's semicircular canal 126. Because, as noted above, vestibule 129 (FIG. 1A) provides fluid communication between the semicircular canal 126 and the median canal 136 (FIG. 1B), the wave of fluid motion continues into median canal 136, thereby activating the hair cells of the organ of Corti 150 (FIG. 1C). Activation of the hair cells causes appropriate nerve impulses to be generated and transferred through the spiral ganglion cells (FIG. 1C) and auditory nerve (FIG. 1A) to the recipient's brain where they are perceived as sound.

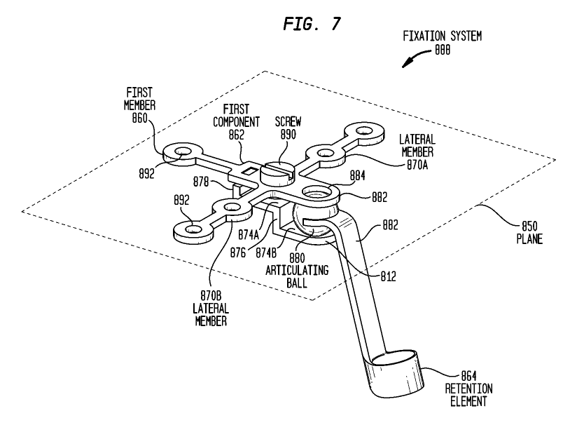

In the illustrative embodiment, stimulation arrangement 450 comprises an actuator 440 coupled to a stimulator unit (not shown) by one or more cables 428. Actuator 440 may be positioned and secured to the recipient by a fixation system. Details of an exemplary fixation system are provided below with reference to FIG. 7. Stimulation arrangement 450 further comprises a stapes prosthesis 452. In the illustrative embodiment, stapes prosthesis 452 is a substantially cylindrical member having a first end 460 abutting an opening 405 in the recipient's horizontal semicircular canal 126.

Connecting actuator 440 and stapes prosthesis 452 is a coupler 409. Coupler 409 comprises a first elongate component 404 extending longitudinally from actuator 440. Disposed at the distal portion of first component 404 is a second component 406. Second component 406 is oriented such that the component extends away first component 404 at an angle and connects to stapes prosthesis 452. In other words, an axis 411 extending through the center of second component 406 along the direction of orientation is at an angle from the longitudinal axis 407 of first component 404. In certain embodiments, second component 406 is oriented such that axis 411 is positioned at an angle of approximately 125 degrees from longitudinal axis 407.

As would be appreciated, there is limited space within a recipient's skull in which stimulation arrangement 450 may be implanted particularly if the recipient's middle ear is left undisturbed. As such, due to these size constraints the orientation of second component 406 relative to first component 404 may facilitate the proper or desired positioning of stapes prosthesis 452 to optimally mechanically stimulate the recipient. To implant stimulation arrangement 450 illustrated in FIG. 4A, a surgeon may drill or form a passageway in the mastoid of the skull. This passageway is preferably constructed and arranged such that it provides direct access to the cochlea. In this embodiment, the surgeon then drills or forms an opening in semicircular canal 126 of the recipient. Stimulation arrangement 450 may be implanted in the formed passageway and/or the recipient's middle ear cavity, and the arrangement is configured so that stapes prosthesis 452 is positioned abutting the opening in the semicircular canal 126. In the illustrative embodiment of FIG. 4A, this opening is created in horizontal semicircular canal 126. It would be appreciated that an opening created in posterior semicircular canal 127 (FIG. 1A) or superior semicircular canal 128 (FIG. 1A) may also be used.

In embodiments of the present invention, first component 404 comprises an elongate rod 404. FIG. 4B illustrates one exemplary configuration for a rod 404. As shown in FIG. 4B, rod 404 comprises a plurality of telescoping sections 420 configured to be slidably engaged with one another. As used herein, telescoping sections refer to sections that can slide inward or outward with respect to each other. The telescoping sections 420 have increasing cross-sectional diameters, such that each telescoping section may be received within an adjacent larger telescoping section. As noted above, due to size constraints, there may be limited locations in which actuator 440 may be implanted. Telescoping sections 420 enhances the adjustment capabilities within the limited space provided in a recipient's skull so that the stapes prosthesis may be properly positioned at the opening in semicircular canal 126.

In the specific embodiment of FIG. 4B, rod 404 comprises three sections 420. First section 420A has the largest cross-sectional diameter and sections 420B and 420C have increasing smaller cross-sectional diameters. Rod 404 is constructed and arranged such that each section 420 may be independently retracted or extended so as to permit various lengths of rod 404. For example, if a shorter rod 404 is desired in one configuration, sections 420B and 420C may be both retracted into section 420A. In other embodiments, section 420B may be extended from section 420A, while section 420C remains in a retracted positioned within 420B. Sections 420 include interlocking mechanisms which independently lock the sections in a desired retracted or extended configuration.

Although FIG. 4B has been discussed herein with reference to three telescoping sections 420, it would be appreciated that the use of greater or lesser numbers of sections is within the scope of the present invention. Furthermore, although telescoping sections 420 are illustrated as having a cylindrical cross-sectional shape, it should be understood that in other embodiments the telescoping sections may have different cross-sectional shapes, such as, for example, rectangular, triangular, etc.

As noted above, second component 406 is attached to a distal portion of first component 404 and extends there from at an angle. In embodiments of the present invention, second component 406 is attached to first component 404 so as to extend there from at a predetermined angle. In other embodiments, second component 406 is attached to first component 404 by a pivot joint which permits adjustment of the angle of orientation of the second component. FIG. 4C is a cross-sectional view of an exemplary second component 406 connected to first component 404 by a pivot joint 436. In the illustrative embodiment, pivot joint 436 comprises a ball 434 and a socket 430, collectively referred to as ball and socket joint 436 herein. Ball 434 is disposed at the distal end of first component 434 and is configured to be received in socket 430 of second component 406. As shown, the center of ball 434 is positioned at longitudinal axis 407 of first component 404. Ball and socket joint 436 is constructed and arranged such that socket 430 may be rotated about longitudinal axis 404 or along longitudinal axis 404. This provides two degrees of freedom in the adjustment of the angle of second component 406.

As shown, ball and socket joint 436 may further comprises a locking arrangement 442. Once a desired angle of second component 406 has been set, locking arrangement 442 may be engaged to retain the second component in the desired configuration.

As noted above, stapes prosthesis 452 is connected to second component 406. FIG. 4C illustrates one exemplary arrangement for connecting stapes prosthesis 452 to second component 406. As shown, second component comprises a receiving member 432 therein. An element disposed at the proximal end of stapes prosthesis 452 is configured to mate with receiving member 432. In certain embodiments, stapes prosthesis 452 is detachable from second component 406. For example, in one embodiment, the proximal element of stapes prosthesis 452 is resiliently flexible and is configured to snap into receiving member 432. In other embodiments, receiving member 432 has threads therein which are configured to mate with threads on the proximal element of stapes prosthesis. It should be appreciated that other connections may also be used in alternative embodiments. In all embodiments, the connection would be constructed and arrangement so as not to interfere with the transmission of vibration from actuator 440 to stapes prosthesis 452.

As noted above, due to size constraints, there may be limited locations in which actuator 440 may be implanted within the recipient. Connecting first and second components 404, 406 in a manner which permits adjustment of the orientation and/or position of stapes prosthesis 452 facilitates optimal positioning of the prosthesis for stimulation.

FIG. 5A illustrates a stimulation arrangement 550 in accordance with embodiments of the present invention. In the illustrative embodiment of FIG. 5A, stimulation arrangement 550 is configured to generate fluid motion of the perilymph contained in a recipient's scala tympani 138 (FIG. 1B). As discussed above, fluid motion of the perilymph activates the hair cells of the organ of Corti 150 (FIG. 1C). Activation of the hair cells causes appropriate nerve impulses to be generated and transferred through the spiral ganglion cells (FIG. 1C) and auditory nerve (FIG. 1A) to the recipient's brain where they are perceived as sound.

In the illustrative embodiment, stimulation arrangement 550 comprises an actuator 540. Actuator 540 may be positioned and secured to the recipient by a fixation system. Details of an exemplary fixation system are provided below with reference to FIG. 7. Stimulation arrangement 550 further comprises a stapes prosthesis 554. As shown in FIG. 5C, stapes prosthesis 554 is a substantially cylindrical member having a first end 560 and a second end 514. As shown, first and second ends 560 and 514 have cross-sectional diameters which exceed the cross-sectional diameter of the remainder of prosthesis 554. Returning to FIG. 5A, distal end 560 is configured to be positioned abutting the membrane of round window 121 in the recipient's cochlea.

Connecting actuator 540 and stapes prosthesis 554 is a coupler 509. Due to size constraints, there may be limited locations in which actuator 540 may be implanted within the recipient, particularly if the recipient's inner ear is to remain undisturbed. FIG. 5A illustrates embodiments in which actuator 540 is positioned substantially in line with round window 121. That is, actuator 540 is positioned along or parallel to an axis extending through the geometric center of round window 121. As such, in this exemplary configuration coupler 509 comprises an elongate rod extending longitudinally from actuator 540 along axis 507. The distal portion of rod 508 is connected to stapes prosthesis 554. In the illustrative embodiment of FIG. 5A, stapes prosthesis 554 is aligned along, and is substantially symmetrical about axis 507. In other words, the surface of first end 560 is positioned orthogonal to axis 507.

FIG. 5D is cross-sectional view of one embodiment of stapes prosthesis 554 illustrating one exemplary arrangement for connecting the stapes prosthesis to rod 509. In the illustrative embodiment, stapes prosthesis 554 has an elongate channel 555 extending at least partially there through. As shown, channel 555 has a cylindrical shape which is symmetrical about axis 507. More specifically, channel 555 is shaped so as to receive at least the distal portion of rod 509 therein. As would be appreciated, the distance between actuator 540 and second end 514 of stapes prosthesis 554 may be increased or decreased bending on the extent to which rod 509 is inserted into channel. Once a desired distance between second end 514 and actuator 540 is reached, rod 509 may be secured within channel 555. For example, in one embodiment rod 509 has threads thereon. In this embodiment, channel 555 has threads therein configured to mate with the threads of rod 509.

In alternative embodiments, channel 555 is configured to constrictably engage rod 509. In one such embodiment, channel 555 is lined with a material which exerts a compressive force on rod 509 when it is inserted into channel 555. This compressive force is sufficient to couple stapes prosthesis 554 to rod 509, but may be low enough that the rod and prosthesis may be manually separated.

As noted, the implanted position of actuator 540 may depend upon the size constraints of a particular recipient's skull. As such, in alternative embodiments of the present invention, actuator 540 may not be positioned along or parallel to an axis extending through the geometric center of round window 121. Therefore, in certain embodiments, coupler 509 may be implemented in one of the configurations described above with reference to FIG. 4A. For example, in certain embodiments, coupler 509 may comprise telescoping sections, a ball and socket joint, etc.

FIG. 5B illustrates an alternative configuration for stimulation arrangement 550. In this embodiment, stimulation arrangement 550 is configured to generate fluid motion of the endolymph contained in a recipient's semicircular canal 126. Because, as noted above, vestibule 129 (FIG. 1A) provides fluid communication between the semicircular canal 126 and the median canal 136 (FIG. 1B), the wave of fluid motion continues into median canal 136, thereby activating the hair cells of the organ of Corti 150 (FIG. 1C). Activation of the hair cells causes appropriate nerve impulses to be generated and transferred through the spiral ganglion cells (FIG. 1C) and auditory nerve (FIG. 1A) to the recipient's brain where they are perceived as sound.

As discussed above, in these embodiments, stimulation arrangement 550 comprises an actuator 540. Actuator 540 may be positioned and secured to the recipient by a fixation system. Details of an exemplary fixation system are provided below with reference to FIG. 7. Stimulation arrangement 550 further comprises a stapes prosthesis 554. As shown in FIG. 5C, stapes prosthesis 554 is a substantially cylindrical member having a first end 560 and a second end 514. As shown, first and second ends 560 and 514 have cross-sectional diameters which exceed the cross-sectional diameter of the remainder of prosthesis 554. Returning to FIG. 5A, distal end 560 is configured to be positioned abutting an opening in semicircular canal 126.

Connecting actuator 540 and stapes prosthesis 554 is a coupler 509. Due to size constraints, there may be limited locations in which actuator 540 may be implanted within the recipient, particularly if the recipient's inner ear is to remain undisturbed. FIG. 5A illustrates embodiments in which actuator 540 is positioned along or parallel to an axis extending through the geometric center of the opening in semicircular canal 126. As such, in this exemplary configuration coupler 509 comprises an elongate rod extending longitudinally from actuator 540 along axis 507. The distal portion of rod 508 is connected to stapes prosthesis 554. In the illustrative embodiment of FIG. 5A, stapes prosthesis 554 is aligned along, and is substantially symmetrical about axis 507. In other words, the surface of first end 560 is positioned orthogonal to axis 507. Stapes prosthesis 554 may be connected to coupler 509 as described above with reference to FIG. 5A.

As noted, the implanted position of actuator 540 may depend upon the size constraints of a particular recipient's skull. As such, in alternative embodiments of the present invention, actuator 540 may not be positioned along or parallel to an axis extending through the geometric center of the opening in semicircular canal 126. Exemplary such embodiments are illustrated in FIG. 4A.

The adjustment in the length provided by the above configuration allows stimulation arrangement 550 to be adjusted for use in a particular recipient, without having to manufacture different length rods 509 and stapes prosthesis 554. In other embodiments, rod 509 may comprise a plurality of telescoping sections, such as described above with reference to FIG. 4B to provide adjustment in the length.

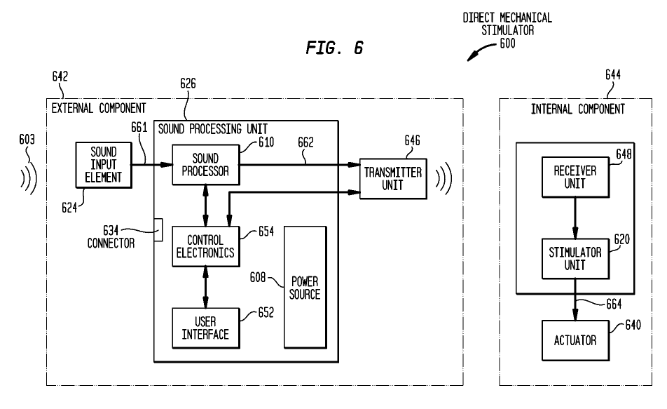

FIG. 6 is a functional block diagram of a direct mechanical stimulator 600 in accordance with embodiments of the present invention. As shown, direct mechanical stimulator 600 comprises an external component 642 and an internal component 644. External component 642 comprises one or more sound input elements 624, a sound processing unit 626, a power source 620, and an external transmitter unit 631.

Sound input element 624 receives a sound 603 and outputs an electrical signal 661 representing the sound to a sound processor 610 in sound processing unit 626. Sound processor 610 generates encoded signals 662 which are provided to external transmitter unit 646. As should be appreciated, sound processor 610 uses one or more of a plurality of techniques to selectively process, amplify and/or filter electrical signal 661 to generate encoded signals 662. In certain embodiments, sound processor 610 comprises substantially the same sound processor as is used in an air conduction hearing aid. In further embodiments, sound processor 610 comprises a digital signal processor.

External transmitter unit 646 is configured to transmit the encoded data signals to internal component 644. In certain embodiments, external transmitter unit 646 comprises an external coil which forms part of a radio frequency (RF) link with components of internal component 644.

Internal component 644 comprises an internal receiver unit 648, a stimulator unit 620, and a stimulation arrangement which includes an actuator 640. Internal receiver unit 648 comprises an internal coil which receives power and encoded signals from the external coil in external transmitter unit 646. The encoded signals 662 received by internal receiver unit 633 are provided to stimulator unit 620. Based on the received signals, stimulator unit 620 is configured to deliver an electrical drive signal 664 to actuator 640. Based on drive signal 664, actuator 640 vibrates a component abutting an opening in a recipient's inner ear to generate fluid motion of the cochlear fluid.

As shown in FIG. 6, sound processing unit 626 further comprises a user interface 652 and control electronics 654. These components may function together to permit a recipient or other user of direct mechanical stimulator 600 to control or adjust the operation of the stimulator. For example, in certain embodiments of the present invention, based on inputs received by a user interface 652, control electronics 654 may provide instructions to, or request information from, other components of direct mechanical stimulator 600. User interface 652 may comprise one or one or buttons or inputs which allow the recipient to adjust the volume, alter the speech processing strategies, power on/off the device, etc.

Although the embodiments of FIG. 6 have been described with reference to an external component, it should be appreciated that in alternative embodiments direct mechanical stimulator 600 is a totally implantable device. In such embodiments, sound processing unit 626 is implanted in a recipient in the mastoid bone. In such embodiments, sound processor may communicate directly with stimulator unit 620 and the transmitter and receiver may be eliminated.

FIG. 7 is a perspective view of a fixation system 888 implemented in conjunction with a direct mechanical stimulator in accordance with embodiments of the present invention. Fixation system 888 is configured to be implanted, for example, in the middle ear cavity of the recipient in order to retain a stimulation arrangement in a desired positioned. As noted, the size constraints of a particular recipient's skull may limit how components of a mechanical stimulator may be positioned within a recipient. As described below, fixation system 888 provides a flexible system that permits fixation of an actuator in a number of positions within a recipient. Such a flexible system provides the ability to customize the stimulation arrangement for optimal cochlear fluid displacement within the geometric size constraints of the middle ear.

As shown, fixation system 888 first comprises a first cross-shaped component 860. First component 860 comprises a first elongate and substantially planar member 862 positioned in a plane 850. Extending laterally from first member 860 in plane 850 are symmetrical members 870. First member 860 and lateral members 870 each have one or more apertures 892 therein used to secure the fixation system to the recipients skull. Specifically, during implantation of fixation system 888, one or more bone screws (not shown) are drilled into the recipient's skull through apertures 892. The screws exert a force on component 860 which secures the component in a selected positioned.

Coupled to first component 860 is a second component 872. Second component 872 comprises first and second planar portions 874 positioned substantially parallel to plane 850. Portions 874 are separated by an orthogonal member 876 positioned orthogonal to plane 850. As shown in FIG. 7, portion 874A is positioned adjacent to first member 860 and secured thereto by a screw 890. Portion 874 is spaced from first member 860 by a spacer 878.

Similar to portion 874A, portion 874B is positioned parallel to a portion 882 of first member 860. Portion 874B is spaced from portion 882 by spacer 878 and orthogonal member 876. As shown in FIG. 7, portions 874B and 882 each comprise an aperture 884 dimensioned to receive a spherical element 880, referred to herein articulating ball 880, therein. The diameters of apertures 884 are smaller than the diameter of articulating ball 880 such that only a portion of the ball is received therein. As discussed above, screw 890 secures first component 862 to second component 872. Screw 890 serves a second purpose of securing the position of articulating ball 880. Specifically, as screw 890 is tightened, portions 882 and 874B are forced together. This exerts a compressive force on articulating ball 888 which prevents any rotation of the ball within apertures 884.

Affixed to and extending from articulating ball 880 is an L-shaped elongate member 880. Disposed at the distal end of elongate member 880 is an actuator retention element 864. Actuation retention element 864 comprises a hollow tube which is configured to receive and retain the body of an actuator therein. Retention element 864 is configured to securely hold an actuator therein during mechanical stimulation of a recipient's inner ear. As would be appreciated, other types of retention elements are within the scope of the present invention. For example, in one embodiment, the actuator comprises a metallic outer body. In such an embodiment, retention element 864 may comprise a magnet configured to create a magnetic connection with the outer body of the actuator.

As noted above, during implantation of a of fixation system 888, one or more bone screws are drilled into the recipient's skull through apertures 892 to secure the system to the recipient. Prior or subsequent to implantation, screw 890 is adjusted to such that articulating ball 880 is free to rotate in apertures 884. By proving freedom of movement of articulating ball 880, a surgeon may adjust the location, position and/or orientation of retention element 864 in any axis. This freedom of movement provides the surgeon with the ability to precisely position retention element 864 such that an actuator received therein will be properly positioned to transfer vibration to a stapes prosthesis positioned at various locations in the inner ear.

In embodiments of the present invention, elongate member 880 may have an adjustable length. For example, in one such embodiment, elongate member 880 may comprise a plurality of telescoping sections configured to be slidably engaged with one another. As used herein, the term telescoping sections refers to sections that can slide inward or outward with respect to each other. The telescoping sections have increasing cross-sectional diameters, such that each telescoping section may be received within an adjacent larger telescoping section.

In other embodiments, the location of retention element 864 is adjustable. For example, in one retention element 864 is mounted on a rail system. In such an embodiment, retention element 864 would be configured to slide along the rail into a desired location. The rail system would be configured to lock retention element 864 into the desired location.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A mechanical stimulator for evoking a hearing percept by directly generating waves of fluid motion of fluid in a recipient's scala tympani, comprising:
a sound processing unit configured to process a received sound signal; and
an implantable stimulation arrangement, comprising:
an actuator configured to receive electrical signals representing the processed sound signal and configured to vibrate in response to the electrical signals,
a stapes prosthesis having first and second ends, the first end having a surface configured to be positioned abutting the round window in the recipient's cochlea, and wherein the first end surface is substantially orthogonal to a longitudinal axis extending through the actuator,
an elongate rod extending longitudinally from the actuator connecting the actuator to the stapes prosthesis such that vibration of the actuator results in waves of fluid motion in a recipient's scala tympani that evoke a hearing percept of the received sound signal.

2. The mechanical stimulator of claim 1, wherein the stapes prosthesis further comprises:
an elongate channel at least partially extending there through from the second end, wherein the channel is configured to receive a portion of the elongate rod therein.

3. The mechanical stimulator of claim 2, wherein the elongate rod has threads thereon, and wherein the channel has threads therein configured to mate with the threads of the elongate rod.

4. The mechanical stimulator of claim 2, wherein the channel is configured to constrictably engage the elongate rod.

5. The mechanical stimulator of claim 1, wherein the elongate rod has an adjustable length.

6. The mechanical stimulator of claim 5, wherein the elongate rod comprises:
a plurality of telescoping sections slidably engaged with one another, each section movable between a retracted configuration and an expanded configuration.

7. The mechanical stimulator of claim 1, wherein the stapes prosthesis comprises:
an elongate cylindrical member, wherein the first end of the member has a surface area which is larger than the surface area of the round window.

8. The mechanical stimulator of claim 1, wherein the first end of the stapes prosthesis is permanently secured to the round window, and wherein the stapes prosthesis is detachably connected to the coupler.

9. The mechanical stimulator of claim 1, wherein the actuator includes a piezoelectric transducer.

10. The mechanical stimulator of claim 1, further comprising:
a sound input element configured to receive a sound signal, wherein the sound processing unit is configured convert the received sound signal into encoded data signals.

11. The mechanical stimulator of claim 10, wherein the sound input element and the sound processing unit are configured to be positioned external to the recipient, and wherein the mechanical stimulator further comprises:
an internal receiver unit configured to be implanted in the recipient;
an external transmitter unit configured to receive the encoded data signals from the sound processing unit and to transmit the encoded data signals to the receiver unit; and
a stimulator unit configured to generate electrical signals configured to cause vibration of the actuator that results in waves of fluid motion in a recipient's scala tympani that evoke a hearing percept of the sound signal received at the sound input element.

12. The mechanical stimulator of claim 10, wherein the sound input element and the sound processing unit are implantable in the recipient.

13. A system for rehabilitating the hearing of a recipient, comprising:
a sound processing unit configured to process a received sound signal;
an actuator configured to receive electrical signals representing the processed sound signal and configured to vibrate in response to the electrical signals;
a stapes prosthesis having a first end configured to be positioned abutting the round window in a recipient's cochlea;
an elongate rod extending from the actuator; and
a fixation system configured to be attached to the actuator and configured to position the actuator such that the coupler connects the actuator to the stapes prosthesis so that vibration of the actuator results in waves of fluid motion in the recipient's semicircular canal that evoke a hearing percept of the received sound signal.

14. The system of claim 13, wherein the fixation system comprises:
a first component configured to be affixed to the recipient;
a second component secured to the first component by a screw;
an articulating ball positioned and retained between the first and second components;
an elongate member attached to and extending from the articulating ball; and
an actuator retention element disposed at the distal end of the elongate member,
wherein adjustment of the screw permits manipulation of the articulating ball.

15. The system of claim 14, wherein the actuator has a cylindrical outer body, and wherein the retention element comprises:
a hollow tube configured to receive and retain the cylindrical body of the actuator therein.

16. The system of claim 14, wherein the actuator has a metallic outer body, and wherein the actuator retention element comprises:
a magnet configured to create a magnetic connection with the metallic outer body of the actuator.

17. The system of claim 14, wherein the elongate member extending from the articulating ball has an adjustable length.

18. The system of claim 14, wherein the position of the actuator retention element is adjustable along the length of the elongate member.

19. The system of claim 13, wherein the stapes prosthesis further comprises:
an elongate channel at least partially extending there through from the second end, wherein the channel is configured to receive a portion of the elongate rod therein.

20. The system of claim 19, wherein the elongate rod has threads thereon, and wherein the channel has threads therein configured to mate with the threads of the elongate rod.

21. The mechanical stimulator of claim 19, wherein the channel is configured to constrictably engage the elongate rod.

22. The mechanical stimulator of claim 13, wherein the elongate rod has an adjustable length.

23. The mechanical stimulator of claim 22, wherein the elongate rod comprises:
a plurality of telescoping sections slidably engaged with one another, each section movable between a retracted configuration and an expanded configuration.

24. The system of claim 13, further comprising:
a sound input element configured to receive a sound signal, wherein the sound processing unit is configured convert the received sound signal into encoded data signals.

25. The system of claim 24, wherein the sound input element and the sound processing unit are positioned external to the recipient, and wherein the system further comprises:
an internal receiver unit configured to be implanted in the recipient;
an external transmitter unit configured to receive the encoded data signals from the sound processing unit and to transmit the encoded data signals to the receiver unit; and
a stimulator unit configured to generate electrical signals configured to cause vibration of the actuator that results in waves of fluid motion in a recipient's semicircular canal that evoke a hearing percept of the sound signal received at the sound input element.

26. The system of claim 24, wherein the sound input element and the sound processing unit are implantable in the recipient.

27. A method for rehabilitating the hearing of a recipient using a mechanical stimulator comprising a sound input element, a sound processing unit and an implantable stimulation arrangement, the method comprising:
receiving at the sound input element an acoustic sound signal;
converting with the sound processing unit the received sound signal into encoded data signals representing the received sound signal;
providing the encoded data signals to the implantable stimulation arrangement; and
generating with the implantable stimulation arrangement waves of fluid motion in a recipient's scala tympani that evoke a hearing percept of the received sound signal

28. The method of claim 27, wherein the stimulation arrangement comprises a stapes prosthesis having a first end configured to be positioned abutting the round window in a recipient's cochlea, an actuator, and an elongate rod connecting the actuator to the stapes prosthesis, wherein generating the waves of fluid motion the method further comprises:
receiving at the actuator electrical signals representing the processed sound signals;
generating vibration with the actuator based on the electrical signals; and
delivering with the stapes prosthesis the vibration to round window.

29. The method of claim 28, wherein the sound input element and the sound processing unit are positioned external to the recipient, and wherein the mechanical stimulator further comprises an internal receiver unit configured to be implanted in the recipient, an external transmitter unit, and a stimulator unit, wherein the method further comprises:
transmitting the encoded signals from the external transmitter unit to the internal receiver unit;
delivering to the stimulator unit the encoded signals received by the internal receiver unit;
generating with the stimulator unit electrical signals representing the encoded signals;
delivering the electrical signals representing the encoded signals to the actuator.
